(19) United States
(12) Patent Application Publication
(10) Pub. No.: US 2006/0216728 A1
(43) Pub. Date: Sep. 28, 2006

(54) Method of fabricating biochip

(76) Inventors: Youngduk Kim, Daejeon-city (KR); Eunjeong Lee, Yeosu-city (KR); Dong Jo Ryu, Daejeon-city (KR); Jae Kwon Kim, Goyang-city (KR); Jaeyoung Jang, Daejeon-city (KR)

Correspondence Address: MCKENNA LONG & ALDRIDGE LLP;Song K. Jung, 1900 K Street, N.W., Washington, DC, 20006, US

(21) Appl. No.: 11/301,244

(22) Filed: Dec. 13, 2005

(30) Foreign Application Priority Data
Dec. 13, 2004 (KR) 10-2004-0104944

(51) Int. Cl. C12Q 1/68

(52) U.S. Cl. 435/6

(57) ABSTRACT

Provided is a method of fabricating a biochip using microspotting, the method including immobilizing probes or probe-attached beads on a surface of a substrate using an adhesive.

This application claims the priority of Korean Patent Application No. 10-2004-0104944, filed on Dec. 13, 2004, in the Korean Intellectual Property Office, the disclosure of which is incorporated herein in its entirety by reference.

### BACKGROUND OF THE INVENTION

1. Field of the Invention

The present invention relates to a method of fabricating a biochip using microspotting, and more particularly, to a method of immobilizing an integrated array of probes or probe-attached beads on a surface of a substrate of a biochip.

2. Description of the Related Art

Biochips are devices with various kinds of probes immobilized on the surface of a chip substrate. Using such biochips, diagnoses of diseases and experiments, such as high throughput screening (HTS), enzyme assay, etc., can be easily performed on a large scale using small amounts of samples.

Most methods of immobilizing probes on a substrate use a glass substrate having a surface pretreated with coating materials. For such surface-treated substrates, various surface chemical species have been suggested, and a technology using self-assembled monolayers has been widely used (Korean Patent Laid-open No. 2003-0038932).

In regard this, to immobilize more probes on the substrate and maintain the activity of the probes, a three-dimensional immobilization method has been developed (Gill. I. and Ballesteros, Trends in Biotechnology 18:282, 2000). For example, a Hydrogel™ coating slide from Packard Bioscience, which has been recently undertaken by PerkinElmer, a polyethylene glycol hydrogel from Biocept. Inc, a Solgel from LG Chem, etc., are used for a three-dimensional immobilization method.

In particular, the Hydrogel™ coating slide from Packard Bioscience utilizes a technology using a 3-dimensinoal polyacrylamide gel. Optically flat silanized Swiss glass used as a base substrate material is surface-modified with acrylamide polymer to enhance protein binding and maintain the three-dimensional structure preserving the activity of protein.

According to the above-described methods, probes are encapsulated into a 3-dimensional gel microstructure maintaining the activity of the probes in spots of samples. However, such a 3-dimensional hydrogel structure has pores of tens of nanometers in size, and thus requires a special mixing device, etc. for analysis. In addition, it takes time to sufficiently transfer biomolecules into the gel of probes, such as protein or DNA. In particular, these limitations are more serious when supplying a target biomolecule into a microchannel of a lab-on-a-chip.

To overcome these drawbacks, a method of immobilizing probes on a substrate via beads having sizes from tens of nanometers to several micrometers has been suggested (K. Sato, Adv Drug Deliv Rev., 55, 379 (2003); H. Andersoon, Electrophoresis, 22, 249 (2001); J.-W. Choi, Biomed. Microdevices, 3, 191 (2001)). According to this method, target probes are immobilized on a solid bead support and then introduced into a microchannel of a lab-on-a-chip. The large surface area of 3-demensional beads can be used as an immobilizing surface area so that more biomolecules can be immobilized. Since solid beads, which are easy to handle, are used, chip processibility is improved.

Representative methods of applying or immobilizing beads on a lab-on-a-chip include a method of trapping a bead in a microchannel and a method of using magnetic fields (K. Sato, Adv Drug Deliv Rev., 55, 379 (2003); H. Andersoon, Electrophoresis, 22, 249 (2001); J.-W. Choi, Biomed. Microdevices, 3, 191 (2001)). In the method of trapping a bead to form a physical barrier in a microchannel of a lab-on-a-chip, the size of the bead is limited to tens of microns or larger to prevent loss of the bead. Therefore, this method is unsuitable for manufacturing a lab-on-a-chip on which a predetermined amount of biological probes has to be immobilized. In the method of using magnetic fields, magnets are installed inside or outside a chip, which inhibits chip miniaturization. In addition, the use of opaque magnetic beads causes limitations in optical measurement.

Methods of introducing or fixing a bead in a microchannel of a lab-on-a-chip include a method of using ultrasonic waves or a laser tweezer. However, this method does not facilitate the manufacturing of a low-cost, miniaturized lab-on-a-chip (A. Meng, Transducers, Sendai, Japan, 876 (1999); K. Dorre, Bioimaging, 5, 139 (1997)).

Therefore, a method of directly immobilizing a bead inside or outside a biochip without using an additional external device is required.

When immobilizing a bead on a substrate in a biochip without using an external device, electrical binding between the bead and the substrate surface, chemical binding between the bead and the substrate surface, or biochemical binding between the substrate and the bead to which biochemical materials, such as biotin-streptavidin, are respectively bound, have to be considered (H. Andersson, Electrophoresis, 22, 3876 (2001); 1. Place, Langmuir, 16, 9042 (2000)). However, in most cases, the binding is not sufficiently strong, and the bead separates from the substrate surface. In addition, to ensure strong binding, the substrate must be processed using plasma, UV light, etc., or a chemical treatment has to be performed to activate the surface of the substrate, which are complicated processes.

### SUMMARY OF THE INVENTION

The present invention provides a method of fabricating a biochip using microspotting, the method including fixing probe-attached beads to a surface of a substrate without chemical activation of the substrate.

The present invention also provides a method of immobilizing probes not attached to beads on a surface of a substrate.

According to an aspect of the present invention, there is provided a method of fabricating a biochip using microspotting, the method comprising immobilizing probes or probe-attached beads on a surface of a substrate using an adhesive.

The immobilization of the probes or probe-attached beads on the substrate may comprise: preparing an aqueous probe or probe-attached bead suspension containing probes or probe-attached beads in an aqueous medium; preparing a water dispersing adhesive containing an aqueous medium, an aqueous adhesive, and an emulsifier; mixing the aqueous probe or probe-attached bead suspension and the water dispersing adhesive to obtain a mixture; and spotting the mixture of the aqueous probe or probe-attached bead suspension and the water dispersing adhesive onto the substrate to immobilize the probes or probe-attached beads on the surface of the substrate.

A method used to spotting the mixture of the aqueous probe or probe-attached bead suspension and the water dispersing adhesive may be, but is not limited to, ink jetting. Any commonly used spotting method can be used.

In the preparing of the water dispersing adhesive, a main monomer selected from the group consisting of butadiene, ethyl acrylate, butyl acrylate, ethylhexyl acrylate, octyl acrylate, and a mixture thereof, a comonomer selected from the group consisting of vinyl acetate, acrylonitrile, acrylamide, styrene, methacrylate, methylacrylate, and a mixture thereof, and a hydrophilic monomer may be added as the aqueous adhesive into the aqueous medium together with the emulsifier.

The hydrophilic monomer may be selected from the group consisting of methacrylic acid, acrylic acid, itaconic acid, hydroxyethylmethacrylate, hydroxypropylmethacrylate, acrylamide, glycidylmethacrylate, polyethyleneglycol acrylate, polyethyleneglycol methacrylate, and a mixture of thereof.

In the mixing of the aqueous probe or probe-attached bead suspension and the water dispersing adhesive, a dispersant may be further added. The dispersant can be a water soluble polymer.

Any arbitrary water soluble polymer commonly known to those of ordinary skill in the art can be used as the dispersant. For example, the water soluble polymer may be selected from, but is not limited to, the group consisting of polyacrylic acid, polymethacrylic acid, polyvinyl alcohol, polyvinyl acetate, polyvinyl pyrrolidone, methylcellulose, carboxymethylcellulose, and a mixture thereof.

The substrate on which the probe-attached beads are immobilized may be one of a micro-well, a slide, and a micro-channel of a lab-on-a-chip.

The substrate can be, but is not limited to, a plastic substrate made of a material selected from the group consisting of polymethylmethacrylate (PMMA), polycarbonate (PC), polystyrene (PS), a cyclic olefin copolymer, polynorbonene, a styrene-butadiene copolymer (SBC), and acrylonitrile butadiene styrene.

Alternatively, the substrate can be a substrate made of a material selected from the group consisting of glass, silicon, hydrogel, metal, ceramic, and a porous membrane.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram illustrating a process of spotting a mixture of an aqueous bead suspension and a water dispersing adhesive onto a substrate using ink jetting when fabricating a biochip according to the present invention and the state of spots fixed to the substrate after being dried;

FIG. 2A is a scanning electron microscopic (SEM) photograph showing the morphology of a spot of a dispersion of beads and an adhesive fixed onto a substrate using ink jetting;

FIG. 2B is a SEM photograph showing particles of the adhesive having a size of 100 nm dispersed between 600 nm beads in the spot of FIG. 2A;

FIG. 3A is a graph of signal-to-noise ratio (SNR) resulting from the measurement of autofluorescence of spots jetted onto Corning glasses using the method according to the present invention in Example 2;

FIG. 3B is a graph of SNR resulting from the measurement of autofluorescence of spots jetted onto polymethylmetacrylate (PMMA) slides using the method according to the present invention in Example 2;

FIG. 4 is a graph of SNR resulting from the measurement of fluorescence indicating the occurrence of non-specific protein binding in spots jetted using the method according to the present invention in Example 3;

FIG. 5A is a photograph of the results of scanning fluorescent spots after specific protein binding to the spots formed using adhesives having different glass transition temperatures in Example 4 using the method according to the present invention;

FIG. 5B is a graph of quantitated fluorescence of spots measured after specific protein binding to the spots formed using the adhesives having different glass transition temperatures in Example 4 using the method according to the present invention;

FIG. 6A is a photograph of the results of scanning fluorescent spots after specific protein binding to the spots formed using different concentrations of adhesive in Example 5 using the method according to the present invention;

FIG. 6B is a graph of quantitated fluorescence of spots measured after specific protein binding to the spots formed using different concentrations of adhesive in Example 5 using the method according to the present invention;

FIG. 7A is a graph of autofluorescence of spots formed using water dispersing adhesives in Example 6;

FIG. 7B is a comparative graph of autofluorescence of spots formed using water dispersing adhesives #4 and #5 and a blank (PMMA slide) in Example 6;

FIG. 8 is a graph of protein immobilization efficiency of spots of mixtures of various water dispersing adhesives and anti-mouse IgG-Cy3 measured in Example 7; and

FIG. 9 is a graph of SNR of spots of the mixtures of various water dispersing adhesives and a protein after immunological reaction to the protein in Example 8.

### DETAILED DESCRIPTION OF THE INVENTION

Hereinafter, the present invention will be described in detail.

The present invention relates to a method of immobilizing probes on a substrate, which is a main process in the fabricating of a biochip, and more particularly, to a method of immobilizing probe-attached beads on a substrate using an adhesive.

Beads having a diameter ranging from tens of nanometers to several micrometers can be used in the method according to the present invention. In addition, materials of beads include, but are not limited to, polystyrene, polymethylmethacrylate, cellulose, polyglycidyl methacrylate, etc., which can be appropriately selected according to the types of probes to be immobilized, the character of a substrate material, and the type of the adhesive used to bind beads.

The inventors of the present invention provide a method of immobilizing probe-attached beads on a surface of a substrate using an adhesive, the method including dispersing probe-attached beads in an aqueous medium to prepare an aqueous bead suspension, adding a water dispersing adhesive containing an aqueous adhesive dispersed in an aqueous medium into the aqueous bead suspension and mixing the mixture to obtain a mixture of the probes or probe-attached beads and the aqueous adhesive, and microspotting the mixture onto a surface of a substrate to immobilize the probe-attached beads on the surface of the substrate in three dimensions. In addition, when preparing the mixture of the probe-attached beads and the aqueous adhesive by mixing the aqueous bead suspension and the water dispersing adhesive, a dispersant may be added to effectively maintain the dispersion of the beads and allow uniform and stable jetting of the mixture using microspotting.

The method of immobilizing probe-attached beads on a substrate using an adhesive may include: preparing an aqueous bead suspension containing probes or probe-attached beads in an aqueous medium; preparing a water dispersing adhesive containing an aqueous medium, an aqueous adhesive, and an emulsifier; mixing the aqueous bead suspension and the water dispersing adhesive to obtain a mixture; and spotting the mixture of the aqueous bead suspension and the water dispersing adhesive onto the substrate to immobilize the probes or probe-attached beads on the substrate.

The aqueous bead suspension may be prepared by adding and dispersing probe-attached beads in an aqueous medium. Examples of the aqueous medium include, but are not limited to, any aqueous solvent, for example, water, ethanol, methanol, dimethylformamide (DMF), dimethyl sulfoxide (DMSO), acetone, N-methylpyrrolidone (NMP), etc. However, water is preferred.

To control the drying speed to prevent formation of doughnut-shaped spots and to increase the activity of biological molecules in the spots, a hydrophilic polymer, such as polyethylene glycol, methylcellulose, hydroxylpropyl cellulose, polyvinyl alcohol, polyacrylic acid, etc., may be added into the aqueous bead suspension.

In the preparing of the water dispersing adhesive, the water dispersing adhesive may be obtained by mixing an aqueous adhesive and an emulsifier in an aqueous medium. Examples of the aqueous medium include, but are not limited to, any aqueous solvent, for example, water, ethanol, methanol, DMF, DMSO, acetone, NMP, etc. However, water is preferred.

The aqueous adhesive refers to an adhesive having adhesive properties in an aqueous medium. In the preparing of the water dispersing adhesive, a main monomer, a comonomer, and a hydrophilic monomer are added into the aqueous medium as the adhesive material. Any arbitrary main monomer and comonomer widely known to those of ordinary skill in the art can be used as the main monomer and comonomer, which are base materials, of the aqueous adhesive. Examples of the main monomer include, but are not limited to, butadiene, ethyl acrylate, butyl acrylate, ethylhexyl acrylate, octyl acrylate, and a mixture thereof. Examples of the comonomer include, but are not limited to, vinyl acetate, acrylonitrile, acrylamide, styrene, methyl methacrylate, methylacrylate, and a mixture thereof. The main monomer provides a target material to be adhered with soft and adhesive properties, and the comonomer provides the adhesive with transparency, processibility, etc.

The hydrophilic monomer used to prepare the water dispersing adhesive improves the dispersibility in an aqueous medium and adhesion of the water dispersing adhesive. Any arbitrary hydrophilic monomer widely known to those of ordinary skill in the art can be used. Examples of the hydrophilic monomer include, but are not limited to, methacrylic acid, acrylic acid, itaconic acid, hydroxyethylmethacrylate, hydroxypropylmethacrylate, acrylamide, glycidylmethacrylate, polyethyleneglycol acrylate, polyethyleneglycol methacrylate, and a mixture of thereof.

The water dispersing adhesive prepared using the materials described above is mixed with the aqueous bead suspension prepared prior to the preparation of the water dispersing adhesive. The mixture of the water dispersing adhesive and the aqueous bead suspension is spotted onto a surface of a substrate to immobilize the beads on the substrate. To uniformly immobilize the beads on the surface of the substrate, the beads must be uniformly and stably dispersed in the mixture. In particular, when using beads larger than few hundreds of nanometers, the gravity of the beads is larger than the dispersing ability of the beads, making it difficult to form a uniform dispersion, which affects the uniformity of bead spots. To uniformly disperse the beads in the mixture, a dispersant may be further added when mixing the aqueous bead suspension and the water dispersing adhesive.

Any dispersant known to those of ordinary skill in the art can be used. Examples of the dispersant include, but are not limited to, polyacrylic acid, polymethacrylic acid, polyvinyl alcohol, polyvinyl acetate, polyvinyl pyrrolidone, methylcellulose, carboxymethylcellulose, and a mixture thereof.

After the mixture of the aqueous bead suspension and the water dispersing adhesive is prepared, the mixture is spotted onto a substrate to immobilize the probe-attached beads on the surface of the substrate. The spotting process may be performed using any arbitrary spotting method known to those of ordinary skill in the art, for example, using ink jetting.

Ink jetting makes it easier to accurately jet a desired quantity of the mixture of the aqueous bead suspension and the water dispersing adhesive according to the present invention onto the substrate.

After the spotting, spots of the mixture are dried. The drying may be performed using a method commonly used to fabricate a biochip using microspotting. For examples, the spots of the mixture may be left at room temperature. The optimal drying temperature varies according to materials to be dried. The optimal drying temperature can be 15-35° C. for protein, and 15-90° C. for DNA.

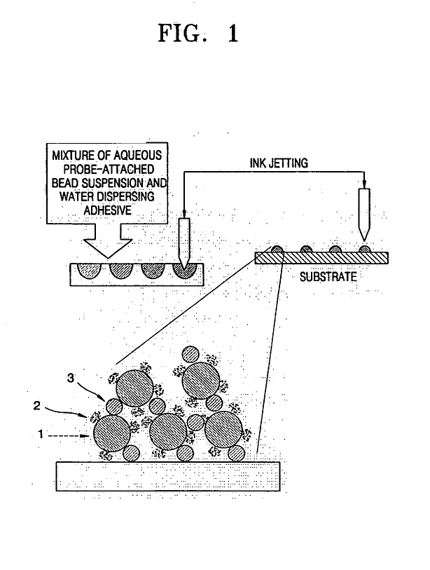

The process of spotting the mixture of the aqueous bead suspension and the water dispersing adhesive onto a substrate using ink jetting and the state of spots fixed to the substrate through the drying process are illustrated in FIG. 1. Referring to FIG. 1, beads 1 with probes 2 attached thereto are immobilized on the substrate via an aqueous adhesive 3. The beads 1 are continuously connected one to another in three dimensions via an aqueous adhesive 3.

In the method of immobilizing probe-attached beads on the substrate according to the present invention, various substrates widely used in the biochip field can be used. Examples of substrates include, but are not limited to, a micro-well, a slide substrate, or a micro-channel of a lab-on-a-chip.

Although substrates made of various materials can be used, a substrate made of a material with a high affinity to the used aqueous adhesive may be used favorably. A substrate made of a material with a high affinity to the used aqueous adhesive can be selected by those of ordinary skill in the art. Examples of the substrate that can be used include, but are not limited to, polymethylmethacrylate (PMMA), polycarbonate (PC), polystyrene (PS), cyclic olefin copolymer, polynorbonene, styrene-butadiene copolymer (SBC), and acrylonitrile butadiene styrene.

In addition, a substrate made of a material selected from the group consisting of glass, silicon, hydrogel, metal, ceramic, and a porous membrane can be used.

In the method according to the present invention, probes are initially attached to beads, and then the beads with the probes are immobilized on the substrate. Due to the large surface area of the beads and the large space between the beads, the method according to the present invention is suitable to immobilize probes, especially in a micro-channel of a lab-on-a-chip. In addition, an array of the bead spots can be effectively used as a platform of a protein chip or a gene chip.

The method according to the present invention provides a technology of spotting tens of spots containing different biological materials onto a single substrate. A biochip fabricated using this method can be used to diagnose various kinds of diseases in a short time using small amounts of samples in the single chip as a substitute for a conventional diagnostic method, such as enzyme immunoassay (EIA). The biochip can be used as a new, high-sensitivity diagnostic tool with thousands to tens of thousands times the sensitivity of conventional devices.

In addition, materials used in the present invention, such as beads, an aqueous adhesive, a dispersant, a plastic substrate, etc., are economical, and the manufacturing costs are low. The method according to the present invention is suitable for mass production of biochips.

The aqueous adhesive used in the present invention may be used to immobilize probes without using beads. In this case, an aqueous solution of probes is mixed with the water dispersing adhesive and then spotted onto a substrate to fabricate a biochip. In this case, rather than being fixed to the surfaces of the beads, probes are immobilized by being three-dimensionally encapsulated in the adhesive.

In a method of fabricating a biochip by spotting a mixture of probes not attached to beads and the water dispersing adhesive onto a substrate, solvents, dispersants, aqueous adhesive, emulsifiers, reaction conditions, etc., described above in connection with the method of fabricating a biochip using the mixture of probe-attached beads and the water dispersing adhesive, can be used.

Hereinafter, the present invention will be described in detail with reference to the following examples. The following examples are only for illustrative purposes and are not intended to limit the scope of the invention.

### Example 1

### Preparation of Water Dispersing Adhesive

167.4 g of deionized water was put into a reactor, and the temperature of the reactor was raised to 75° C. When the temperature of the deionized water reached 75° C., 10.4 g of butylacrylate, 7.7 g of styrene, 0.18 g of sodium lauryl sulfate (SLS) were added to the deionized water. A solution of 0.14 g of potassium sulfate dissolved in 9.0 g of deionized water was added to form polymerized seeds while the temperature of the reactor was maintained at 75° C.

A suspension of 54.0 g of styrene, 108.2 g of butylacrylate, 1.4 g of allyl methacrylate, 9.7 g of itaconic acid, and 0.27 g of SLS in 167.4 g of deionized water and a solution of 0.38 g of potassium sulfate in 18.0 g of deionized water were simultaneously added to the above reactor containing the seeds through several times over 3 hours to obtain a water dispersing adhesive.

### Example 2

### Measurement of Autofluorescence of Spots

To examine whether a water dispersing adhesive to be added to an aqueous bead suspension is autofluorescence, the water dispersing adhesive and the aqueous bead suspension were spotted onto each of a Corning glass and a polymethyl methacrylate (PMMS) slide for tests.

Water dispersing adhesives #1 through #3 were prepared in the same manner as in Example 1 using different kinds and amounts of copolymers and hydrophilic monomers.

Water dispersing adhesive #1 was a mixture containing a copolymer of styrene and butadiene and 10% by weight of itaconic acid and having a glass transition temperature of 0° C. Water dispersing adhesive #2 was a mixture containing a copolymer of styrene and butadiene and 12% by weight of itaconic acid and having a glass transition temperature of 32° C. Water dispersing adhesive #3 was a mixture containing a copolymer of methyl methacrylate and butadiene and 5.6% by weight of itaconic acid and having a glass transition temperature of 30° C. Water dispersing adhesives #4 and #5 were prepared by adding 5.6% by weight of polyethylene glycol and acrylic acid, respectively, instead of itaconic acid and had glass transition temperatures of −20° C. and 10° C., respectively.

Bovine serum albumin (BSA)-coated polystyrene beads having a diameter of 600 nm were used as beads. The bovine serum albumin-coated polystyrene beads were dispersed in a 1×PBS buffer to prepare an aqueous bead suspension.

Mixed solutions 1 through 3 were prepared by mixing the aqueous bead suspension and each of the water dispersing adhesives #1 through #3 to be containing 0.2% by weight of the beads and 0.2% by weight of the adhesive to prepare mixed solutions 1 through 3.

50 nL of each of the mixed solutions 1 through 3 was spotted onto a Corning glass and a PMMA slide, dried at a 70% humidity and 20° C. for one day or longer, and scanned at PMT (Photo mutiplier tube) 600 and 430. The intensity of autofluorescence of spots of each of the mixed solutions 1 through 3 was measured.

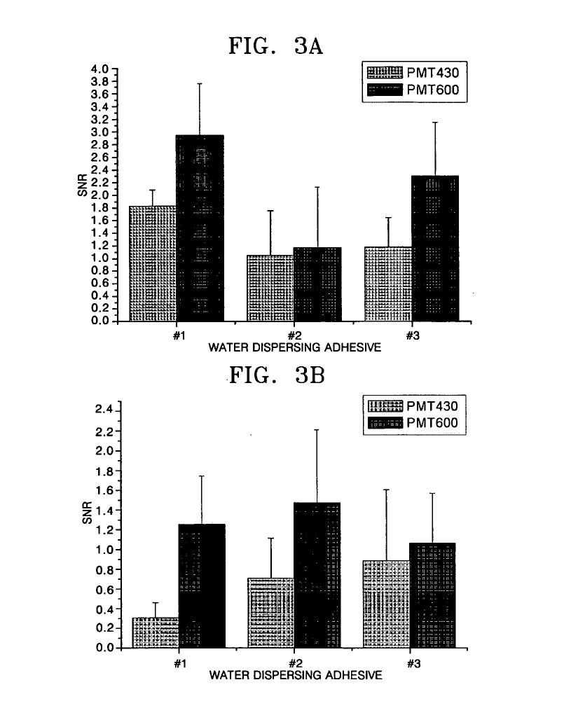

The scanned results are shown in FIGS. 3A and 3B. FIG. 3A is the results for the Corning glasses, and FIG. 3B is the results for the PMMA slides. Referring to FIGS. 3A and 3B, the signal-to-noise (SNR) values of the spots on the Corning glasses and the PMMA slides are not greater than 3 on average at both PMT 600 and 430, indicating that the intensity of autofluorescence of the spots of each of the mixed solutions 1 through 3 is very low.

This result indicates that the method of immobilization probe-attached beads on a substrate using an adhesive according to the present invention can be used to fabricate a biochip because the mixtures of the beads and adhesives do not interfere with fluorescence detection after reaction with a target analyte.

### Example 3

### Non-Specific Protein Binding in Spots

To investigate the degree of occurrence of non-specific protein binding, not caused by an immunological reaction with an antigen or antibody protein, in the bead spots immobilized on the substrate using adhesives, the following experiment was performed.

The bead-spotted PMMA slides in Example 2 were used. A blocking reaction and an incubation with an antibody were performed on each of the PMMA slides in a hybridization chamber, and washing was performed by dipping the PMMA slides in a washing solution in a bath.

In particular, the immunological reaction was performed as follows.

Blocking reaction was performed with PBS containing 1% by weight BSA and 0.05% by weight Tween 20 for 10 minutes. 100 μL of Cy3-labeled (0.01 mg/mL) anti-mouse IgG antibody was added to each of the PMMA slides and reacted for 30 minutes. The PMMA slides were washed using PBS containing 0.05% by weight Tween 20 and then PBS. Since the used Cy3-labeled protein does not cause an immunological reaction with the BSA protein coated on the beads, fluorescent signals detected in the experiment are derived from non-specific protein binding.

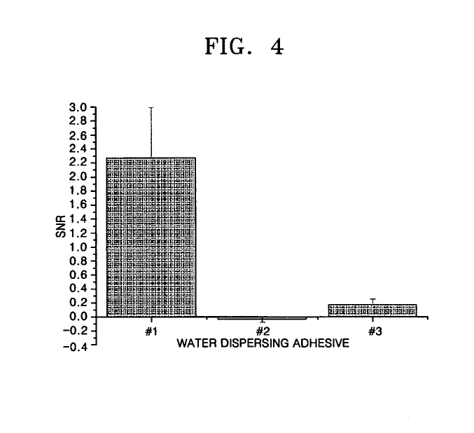

After the reaction, the PMMA slides were scanned using an Axon scanner to quantitate the non-specific protein binding. The results are shown in FIG. 4. Referring to FIG. 4, the non-specific protein binding is almost zero when the water dispersing adhesives #2 and #3 are used. In addition, the SNR value is not greater than 3 when the water dispersing adhesive #1 is used, indicating that the non-specific protein binding is negligible.

From the results described above, it is apparent that only a target analyte, not any other materials, adhere to spots on the substrate in a biochip fabricated using an aqueous adhesive to fix beads to a substrate according to the present invention. That indicates that the water dispersing adhesive does not affect the function of the biochip, and thus the present invention can be used to fabricate a biochip.

### Example 4

### Adhesion Test Using Adhesives Having Different Glass Transition Temperatures

The adhesion of beads to the substrate when adhesives having different glass transition temperatures were used was measured.

As in Example 2, anti-CRP monoclonal antibody-coated bead-spotted PMMA slides were used. An immunological reaction was performed in the same manner as in Example 3, except that Cy3-labeled anti-mouse IgG antibody, which detects the BSA coated on the beads as an antigen, was added.

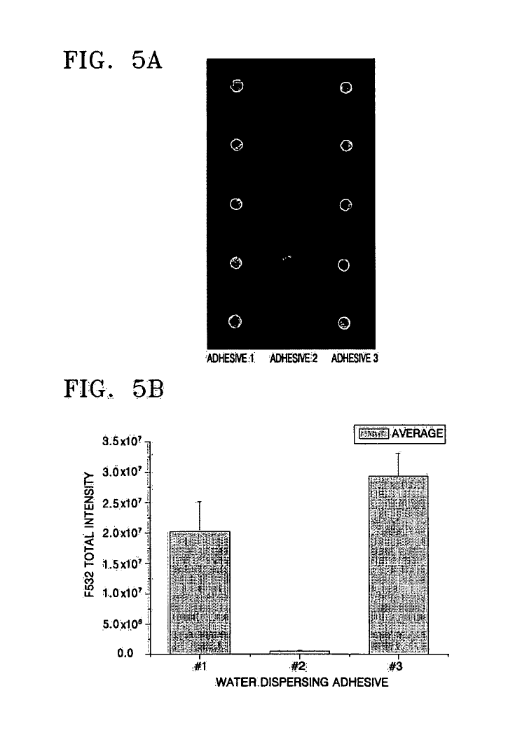

After the immunological reaction, the PMMA slides were scanned using an Axon scanner. A photograph of the scanned results is shown in FIG. 5A, and the quantitated intensities of fluorescence are shown in FIG. 5B.

The results in FIGS. 5A and 5B indicate that the adhesion of beads to the substrate is strong when an adhesive having a glass transition temperature lower than room temperature is used.

### Example 5

### Adhesion Test at Different Adhesive Concentrations

The adhesion of beads to a substrate according to the concentration of a used adhesive was tested as follows.

The mixture of water dispersing adhesive 1 and the aqueous bead suspension in Example 2 was spotted onto a PMMS slide using the same method as used in Example 2 to obtain the bead-spotted substrate. Here, the concentration of the aqueous adhesive was adjusted to be 0.1% by weight, 0.05% by weight, 0.01% by weight, 0.005% by weight, 0.001% by weight, and 0.0005% by weight in the final mixtures to be spotted. The mixtures containing the different concentrations of aqueous adhesive were spotted onto a single substrate. Here, the concentration of beads in each of the mixture was constant at 0.1% by weight.

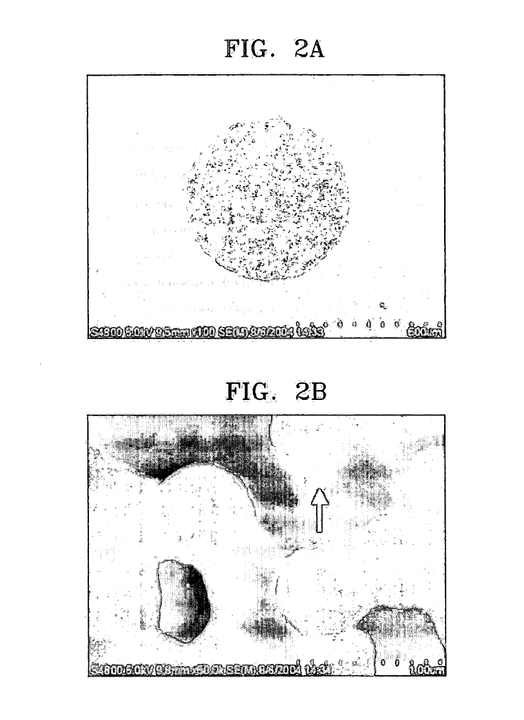

A bead spot containing 0.001% by weight of the adhesive was observed using a scanning electron microscope. The results are shown in FIGS. 2A and 2B. FIG. 2A is a scanning electron microscopic (SEM) photograph of a spot of the dispersion of the beads and the adhesive jetted onto a substrate using ink jetting. FIG. 2B is a SEM photograph of the adhesive having a particle size of 100 nm, dispersed among beads having a size of 600 nm. A 100-nm particle of the adhesive is indicated by an arrow in FIG. 2B.

After the microscopic observation, an immunological reaction was performed using the bead-spotted PMMA slide in the same manner as in Example 4.

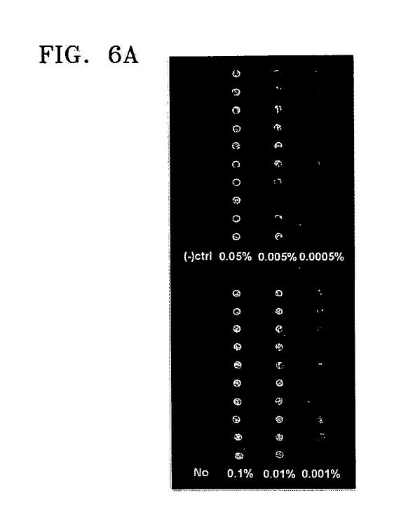

The PMMA slide after the immunological reaction was analyzed using an Axon scanner. A photograph of the scanned results is shown in FIG. 6A, and the quantitated intensities of fluorescence are shown in FIG. 6B.

Referring to FIGS. 6A and 6B, when the ratio of the adhesive to the beads on a % by weight basis is equal to or greater than 1/20, spots of the mixture can be maintained on the substrate. Preferably, when the ratio of the adhesive to the beads is equal to or greater than 1/10, the adhesion of spots to the substrate is maintained, and sufficiently high immunological reaction signals are detected.

### Example 6

### Measurement of Autofluorescence of Adhesive

To investigate whether the adhesive can immobilize a biomolecule without beads, the autofluorescence of the adhesive was measured. Since adhesives form spots with various surface morphologies with respect to polymethylmetacrylate (PMMA), even when mixtures containing different adhesives are spotted at the same volume and the same concentration, the spots have different sizes and shapes. Therefore, to minimize distortion in the measurement of signals due to spot widths, the total intensity of autofluorescence of all of the spots of each of the adhesives was measured.

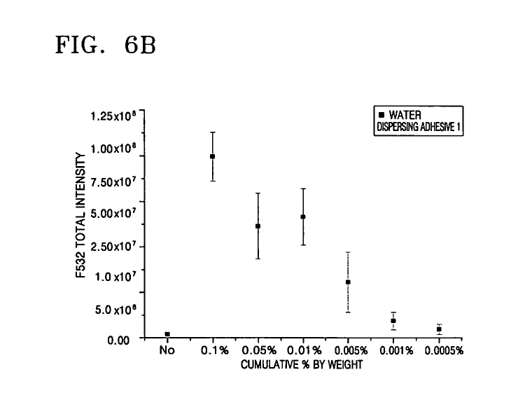

In particular, after measuring the total intensity of spots of each of the water dispersing adhesives, the total intensity of a blank region having the same size as the area of the spots was measured. The total intensity of the blank region was subtracted from the total intensity of spots of each of the water dispersing adhesives. The results of the subtraction are shown in FIG. 7A.

Referring to FIG. 7A, the lowest autofluorescence is exhibited by the water dispersing adhesive 4. The autofluorescence of the water dispersing adhesive 4 is 1.6 times higher than the autofluorescence of the blank region (PMMA) (refer to FIG. 7B).

### Example 7

### Protein Immobilization Efficiency Test

To compare the protein immobilization efficiency between adhesives, 10 ug/mL of anti-mouse IgG-Cy3 was mixed with each of the water dispersing adhesives having a final solid content of 6.4% by weight. Each of the mixtures containing the different water dispersing adhesives was spotted to form an array of 10 spots each having a volume of 10 nL. The arrays were dried at room temperature and a humidity of 50% or greater and washed using 1× phophate buffered saline (PBS) at room temperature for 30 minutes while stirring.

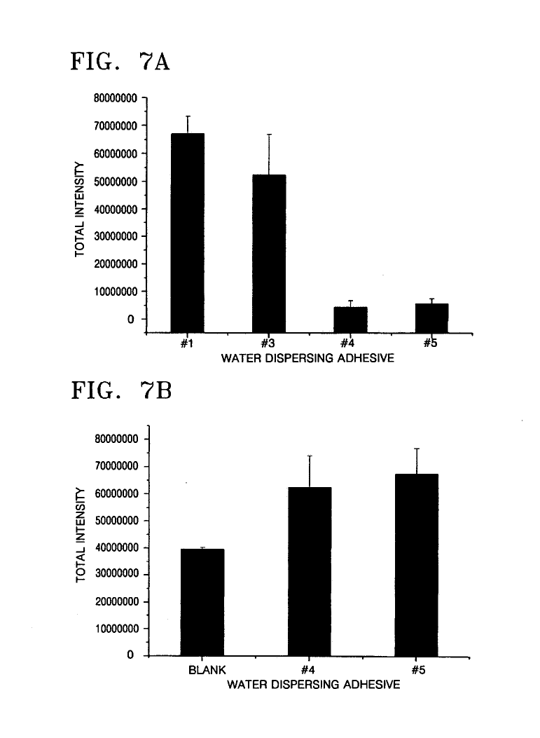

The fluorescence of Cyanine 3 in each of the arrays was measured at 532 nm, and the immobilization efficiency was calculated as follows. The results are shown in FIG. 8.

$$\frac{( Fluorescence after washing - Fluorescence of Blank Region )}{( Fluorescence before washing - Fluorescence of Blank Region )} \times 100$$

Referring to FIG. 8, most of the water dispersing adhesives, except for water dispersing adhesive 3, exhibits an immobilization efficiency of about 80% at the 6.4% adhesive concentration.

### Example 8

### Immonoassay Using Adhesives

An immonoassay was performed using adhesives. In particular, 100 ug/mL of anti-CRP polyclonal IgG was mixed with each of the adhesives having a concentration of 6.4% by weight, spotted as an array of spots, and dried at room temperature and a humidity of 50% or greater in the same manner as in Example 7.

After blocking PMMA slides spotted with the mixtures of each of the water dispersing adhesives 1 through 5 and the protein (anti-CRP polyclonal IgG) using 0.5% by weight BSA in 1×PBS for 5-10 hours, fluorophore Cy-3-conjugated anti-goat polyclonal IgG was spread thereon, reacted at room temperature for 30 minutes, and then washed three times using 1×PBS for 2 minutes each time.

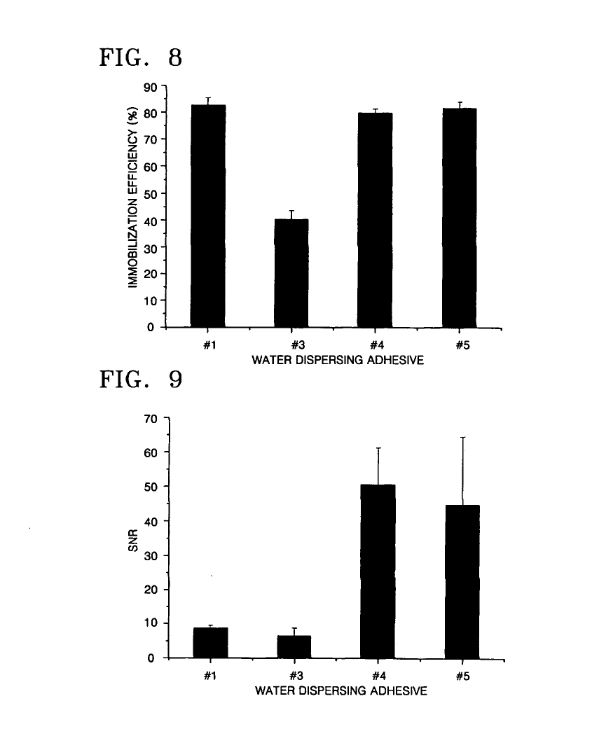

The results of the immunoassay are shown as a signal-to-noise ratio (SNR) in FIG. 9. In particular, the signal level is a value obtained by subtracting the intensity of fluorescence of a control group from the total intensity of fluorescence of the combination of the adhesive and the protein resulting from the immunoassay. The noise level is a value obtained by subtracting the total intensity of a blank from the total intensity of fluorescence of the water dispersing adhesive.

Referring to FIG. 9, the water dispersing adhesive 4 containing polyethylene glycol exhibits the highest SNR. This can be attributed to the increased stability of the fixed protein resulting from the high biocompatibility of the hydrophilic polyethylene glycol polymer.

As described above, in a method of fabricating a biochip according to the present invention, probe-attached beads are directly immobilized on a substrate using an adhesive, not a physical barrier or an electric field. In other words, a small biochip can be manufactured even using beads to immobilize probes on the substrate and economically due to the use of cheap adhesive.

In addition, the method according to the present invention using beads is especially useful to a lab-on-a-chip and can be used to detect various kinds of materials in a short time using small amounts of samples in a single chip and diagnose various diseases in a short time.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of fabricating a biochip using microspotting, the method comprising immobilizing probes or probe-attached beads on a surface of a substrate using an adhesive.

2. The method of claim 1, wherein the immobilizing of the probes or probe-attached beads comprises:
preparing an aqueous probe or probe-attached bead suspension containing probes or probe-attached beads in an aqueous medium;
preparing a water dispersing adhesive containing an aqueous medium, an aqueous adhesive, and an emulsifier;
mixing the aqueous probe or probe-attached bead suspension and the water dispersing adhesive to obtain a mixture; and
spotting the mixture of the aqueous probe or probe-attached bead suspension and the water dispersing adhesive onto the substrate to immobilize the probes or probe-attached beads on the surface of the substrate.

3. The method of claim 2, wherein the spotting of the mixture is performed using ink jetting.

4. The method of claim 2, wherein, in the preparing of the water dispersing adhesive, a main monomer selected from the group consisting of butadiene, ethyl acrylate, butyl acrylate, ethylhexyl acrylate, octyl acrylate, and a mixture thereof, a comonomer selected from the group consisting of vinyl acetate, acrylonitrile, acrylamide, styrene, methyl methacrylate, methylacrylate, and a mixture thereof, and a hydrophilic monomer are added as the aqueous adhesive.

5. The method of claim 4, wherein the hydrophilic monomer is selected from the group consisting of methacrylic acid, acrylic acid, itaconic acid, hydroxyethylmethacrylate, hydroxypropylmethacrylate, acrylamide, glycidyl methacrylate, polyethyleneglycol acrylate, polyethyleneglycol methacrylate, and a mixture of thereof.

6. The method of claim 2, wherein the mixing of the aqueous probes or probe-attached bead suspension and the water dispersing adhesive comprises adding a dispersant.

7. The method of claim 6, wherein the dispersant is a water soluble polymer.

8. The method of claim 7, wherein the water soluble polymer is selected from the group consisting of polyacrylic acid, polymethacrylic acid, polyvinyl alcohol, polyvinyl acetate, polyvinyl pyrrolidone, methylcellulose, carboxymethylcellulose, and a mixture thereof.

9. The method of claim 1, wherein the substrate is one of a micro-well, a slide substrate, and a micro-channel of a lab-on-a-chip.

10. The method of claim 1, wherein the substrate is a plastic substrate made of a material selected from the group consisting of polymethylmethacrylate (PMMA), polycarbonate (PC), polystyrene (PS), a cyclic olefin copolymer, polynorbonene, a styrene-butadiene copolymer (SBC), and acrylonitrile butadiene styrene.
